(19) United States
(12) Patent Application Publication
(10) Pub. No.: US 2010/0132699 A1
(43) Pub. Date: Jun. 3, 2010

(54) BURST VALVE FOR AEROSOL DEVICE

(75) Inventors: Victor BUROLLA, Alameda, CA (US); Henning Graskov, Bagsvaerd (DK); Ling-Kang Tong, Fremont, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: ARADIGM CORPORATION, Hayward, CA (US)

(21) Appl. No.: 12/615,153

(22) Filed: Nov. 9, 2009

Related U.S. Application Data
(63) Continuation-in-part of application No. PCT/US2008/058367, filed on Mar. 27, 2008.
(60) Provisional application No. 60/938,411, filed on May 16, 2007.

(51) Int. Cl. A61M 11/00

(52) U.S. Cl. 128/200.23

(57) ABSTRACT

Aerosol dispensing devices and methods for using the same for administering aerosolized drugs to a subject are provided. Aspects of the aerosol dispensing device include an aerosol production unit configured to produce an aerosol from a liquid containing a pharmaceutically active drug, and a burst valve adapted to mechanically open in response to a subject's inhalation. The burst valve is configured to prevent airflow through the device until the subject's inhalation produces an operational pressure difference across the burst valve. In some embodiments, the operational pressure difference can be 15 cm H2O or less. Aspects of the invention can also include a heating element configured to evaporate water from aerosol droplets. The devices and methods of the invention find use in a variety of applications, such as in applications in which it is desired to administer an aerosolized drug to a subject.

### CLAIM OF PRIORITY

This application is a continuation-in-part of International Patent Application No. PCT/US2008/058367 filed on Mar. 27, 2008; which claims priority to U.S. Provisional Application No. 60/938,411 filed May 16, 2007, the disclosure of which applications are herein incorporated by reference noting that the present application controls to the extent there is a contradiction.

### FIELD OF INVENTION

Embodiments of the present invention relate generally to portable aerosol delivery devices, especially portable aerosol delivery devices for the aerosolized delivery of drugs.

### BACKGROUND OF INVENTION

There are several known methods for the aerosolized delivery of drugs. In general, the methods include systems or portable devices that generate aerosols from liquid formulations. Aerosol delivery of drugs to the lungs has been used for delivery of medication for local (lung) therapy (Graeser and Rowe, Journal of Allergy 6:415 1935). The large surface area, thin epithelial layer, and highly vascularized nature of the peripheral lung (Taylor, Adv. Drug Deliv. Rev. 5:37 1990) also make it an attractive site for non-invasive systemic delivery. However, successful delivery of a pharmaceutical to the peripheral lung requires coordination of a subject's inspiratory effort with production of the aerosolized medication, as well as careful control of the aerosol particle size in order to avoid deposition of the aerosol on the walls of the portable aerosol device, the oropharynx, or the bronchi.

Devices for the intrapulmonary administration of a pharmaceutically active drug, such as a metered dose inhaler, often include a mechanism for actuating a valve within the container to cause release of a metered amount of the drug-containing liquid into a chamber which has a mouthpiece for use by the subject. The mechanism for actuating the valve is often manually operated, (for example, a subject may press on the closed end of the container with a finger to actuate the device). Successful delivery of the medication requires the subject to coordinate the actuation of the valve with inhalation in order to obtain the maximum benefit and optimum distribution of the inhaled drug. However, many subjects who use portable inhalers often have difficulty coordinating their breathing with the manual actuation of the valve, which can result in a subject inadvertently receiving an inadequate or inconsistent dose of the medication.

To overcome this problem, air flow sensors can be used to trigger the production of the aerosol particles only when the air flow caused by the subject's inhalation through the device is sufficient to allow the aerosol particles to effectively pass through the device and reach the peripheral lung of the subject. A constant air flow can also ensure that a consistent drug dose is provided each time the device is used. However, air flow sensors are expensive and can significantly contribute to the cost of portable aerosol delivery devices.

Therefore, there is a need for an inexpensive portable aerosol delivery device which can coordinate delivery of an aerosolized drug with a subject's inspiration, which can increase the effectiveness and efficiency of delivery of a metered dose to the peripheral lungs of a subject.

### SUMMARY OF INVENTION

Aerosol dispensing devices and methods for using the same for administering aerosolized drugs to a subject are provided. Aspects of the aerosol dispensing device include an aerosol production unit configured to produce an aerosol from a liquid containing a pharmaceutically active drug, and a burst valve adapted to mechanically open in response to a subject's inhalation. The burst valve is configured to prevent airflow through the device until the subject's inhalation produces an operational pressure difference across the burst valve. In some embodiments, the operational pressure difference can be 15 cm H₂O or less. Aspects of the invention can also include a heating element configured to evaporate water from aerosol droplets. The devices and methods of the invention find use in a variety of applications, such as in applications in which it is desired to administer an aerosolized drug to a subject.

### BRIEF DESCRIPTION OF THE DRAWINGS

FIG. 1 illustrates an exemplary aerosol delivery device.

FIGS. 2A and 2B illustrate an embodiment of an aerosol delivery device comprising a burst valve.

FIG. 3 illustrates details of burst valve, according to one embodiment of the invention.

### DETAILED DESCRIPTION

Aerosol dispensing devices and methods for using the same for administering aerosolized drugs to a subject are provided. Aspects of the aerosol dispensing device include an aerosol production unit configured to produce an aerosol from a liquid containing a pharmaceutically active drug, and a burst valve adapted to mechanically open in response to a subject's inhalation. The burst valve is configured to prevent airflow through the device until the subject's inhalation produces an operational pressure difference across the burst valve. In some embodiments, the operational pressure difference can be 15 cm H₂O or less. Aspects of the invention can also include a heating element configured to evaporate water from aerosol droplets. The devices and methods of the invention find use in a variety of applications, such as in applications in which it is desired to administer an aerosolized drug to a subject.

Before the present aerosol dispensing device and methods for using the same for administering aerosolized drugs is described, it is to be understood that this invention is not limited to the particular embodiments described, as such aerosol dispensing devices, methods, packages, containers and formulations may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural referents unless the context clearly dictates otherwise. Thus, for example, reference to “a formulation” includes mixtures of different formulations, reference to “an aerosolized compound” includes a plurality of such compounds, and reference to “the method of treatment” includes reference to equivalent steps and methods known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the specific methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “aerosol dispensing device”, “aerosol delivery device” or “drug delivery device” refers to a self contained portable device for the delivery of medication by way of inhalation. The aerosol dispensing device can in some embodiments include a temperature controller component, such as a heating element, and a power source.

The term “aerosol production unit” or “aerosol generation device” refers to any device for forming an aerosol for delivery to a human. These devices include but are not limited to systems that generate aerosols from liquid formulations, such as jet or ultrasonic nebulizers, spinning top generators, devices using an orifice or an array of orifices to form an aerosol (driven by a oscillation mechanism or not). Exemplary liquid-based aerosol production units can force a liquid containing a pharmaceutically active drug through nozzles to create jets containing aerosol particles to be inhaled by a subject.

The term “heating element” refers to an element which can be positioned in an aerosol delivery device in a manner such that air of an aerosol created by the device is warmed when contacting the heating element. The heating element can be capable of converting power provided by a portable power source into heat and releasing it to the surrounding air. In one embodiment the heating element is a metal. The exact structure of the element is not critical, but it must be capable of transferring its heat to the air then to the aerosol over a very short well defined period which may range from 0.1 to 10 seconds, such as from 0.5 to 8 seconds, or from 1 to 2 seconds. In some instances, the heating element is coiled nickel chromium or nickel copper wire, which wire is present in an amount ranging from about 1 to about 10 grams, more preferably about 2-4 grams. If the source of power is a electric cell or group of electric cells (a battery), the heating element must be designed so that its operation is consistent with a battery which is portable (size and weight are small) and can provide enough energy over a short period of time (e.g., one minute or less) to heat the heating element so that it holds enough energy to warm the air into which the aerosol is generated sufficiently to evaporate the desired amount of carrier away from the particles. For example, if the heating element is in the form of a metal wire coil, the wire can not be too thick or too thin, e.g., a nickel chromium wire of about 26±10 gauge.

The heating element can be part of a “portable air temperature controlling device”, or “air temperature controller” which refers to a self-contained device containing a heating element. The device can include a receptacle for a power source for the heating of the heating element, and a control circuit to monitor and control the temperature of the heating element.

The term “receptacle” refers to a location in a portable drug delivery device for connecting a portable power source which power source is preferably two or more electric cells, i.e. a battery. The air temperature controlling device is preferably an integral part of an aerosol delivery device which together (with the power source) weigh less than 1.5 kg; more preferably, less than 0.75 kg. The receptacle may consist of an attachment point essentially outside of the device, or preferably an enclosed volume with a door that contains the power source inside the device. The receptacle can contain a method of connecting and disconnecting the means of transmitting power from the power source to the air temperature controlling device, such as electrical contacts.

The term “portable power source” refers to any source capable of generating power which can be transferred to the heating element in the portable air temperature controlling device, and can be a source of electrical energy, which can in some embodiments be stored in a chemical cell which is an electric cell, e.g., two or more electric cells combined forms a battery. In one embodiment the power source is one or more electrical cells, (i.e. a battery) which is/are sufficiently small such that when loaded into the device the device remains easily portable, e.g., AA size, C size or D size or smaller. Chemical reactions (especially the catalytic combustion of butane), hand-powered generators or friction devices could also be used.

The terms “drug”, “pharmaceutically active drug”, and “active drug” and the like are used interchangeably herein to refer to any chemical compound which, when provided to a mammal, preferably a human, provides a therapeutic effect. The drugs may be drugs intended to provide local therapy to the lungs, and in other embodiments the drugs may be systemic drugs. In some embodiments the drugs can include but are not limited to bronchodilators, anti-inflammatory drugs such as corticosteroids, hormone drugs, peptide hormones, antibodies such as anti-IgE, proteins such as erythropoietin, peptides and the like including insulin and insulin analogs such as insulin lispro, or human recombinant insulin formulated to make the insulin monomeric, enzymes, anticholinergics, antibiotics, antifungals, antivirals, beta-2 agonists, mucolytics, and small molecule drugs including morphine, fentanyl, and the like, i.e. drugs which are commonly used and which are conventionally delivered by injection.

The terms “hormone,” “hormone drug,” “pharmaceutically active hormone formulation,” “peptide used in endocrine therapy,” “peptide hormone drug,” “peptide drug” and the like are used interchangeably herein. A hormone drug as described herein is a peptide drug which has been prepared in a pharmaceutically effective formulation and is useful in endocrine therapy. Specifically, a peptide drug of the type described herein is useful for exogenously modifying the behavior of a subject's endocrine system. The devices and methods disclosed herein can be used in the creation of an aerosol for inhalation into the lungs using any pharmaceutically active peptide. Examples of useful peptides include but are not limited to: Insulin (e.g. human recombinant), Insulin analogs (e.g. insulin lispro), Interferon-alpha, Interferon-gamma, HPTH (human parathyroid hormone), GCSF (granulocyte colony stimulating factor), GMCSF (granulocyte macrophage colony stimulating factor), Atrial natriuretic factor, Angiotensin inhibitor, Renin inhibitor Somatomedin, FSH (follicle stimulating hormone,) Tissue growth factors (TGF's), Endothelial growth factors, HGF (hepatocyte growth factor), Amylin, Factor VIII, and Vasopressin, and IIB/IIIA peptide antagonists, leuprolide, calcitonin, oxyntomodulin, PYY, GLP-1 and nafarelin.

The terms “formulation” and “liquid formulation” and the like are used herein to describe any pharmaceutically active drug by itself or with a pharmaceutically acceptable carrier. The formulation is preferably in flowable liquid form having a viscosity and other characteristics such that the formulation can be aerosolized into particles which are inhaled into the lungs of a subject after the formulation is aerosolized, e.g. by being moved through a porous membrane. Such formulations can be solutions, e.g. aqueous solutions, ethanolic solutions, aqueous/ethanolic solutions, saline solutions, microcrystalline suspensions, liposomal dispersions and colloidal suspensions. Formulations in some embodiments can be solutions or suspensions of drug in a low boiling point propellant and formulations are known which make human recombinant insulin become monomeric.

The term “carrier” shall mean any non-active compounds present in the formulation. The carrier is preferably a liquid, flowable, pharmaceutically acceptable excipient material which the pharmaceutically active drug is suspended in or more preferably dissolved in. Useful carriers do not adversely interact with the drug or packaging and have properties which allow for the formation of aerosol particles having a diameter in the range of 0.5 to 12 microns. The particles may be formed when a formulation including the carrier and drug is forced through pores having a diameter of 0.25 to 3.0 microns. Suitable carriers include but are not limited to water, ethanol and mixtures thereof. Other carriers can be used provided that they can be formulated to create a suitable aerosol and do not adversely affect the drug or human lung tissue. The term carrier includes excipient materials which are used with formulation for nebulizers, and metered dose inhalers or devices of the type described in U.S. Pat. No. 5,709,202.

The term “dosing event” shall be interpreted to mean the administration of a drug to a subject in need thereof by the intrapulmonary route of administration which event may encompass one or more releases of drug formulation from a drug dispensing device over a period of time of 15 minutes or less, preferably 10 minutes or less, and more preferably 5 minutes or less, during which period an inhalation or multiple inhalations are made by the subject and a dose of drug is aerosolized and inhaled. A dosing event can involve the administration of drug to the subject in an amount of ranging from 1 μg to 100 mg of drug from the device. The entire dosing event can involve the administration of anywhere from 10 μg to 1,000 mg of drug formulation, or from 50 μg to 200 μg of drug formulation. This amount of drug is in a liquid form or is dissolved or dispersed within a pharmaceutically acceptable, liquid, excipient material to provide a liquid, flowable formulation which can be readily aerosolized. A container can have a formulation having drug therein in an amount from 10 μg 1 to 300 μg 1, such as 200 μg 1. The large variation in the amounts which might be delivered are due to different drug potencies and different delivery efficiencies for different devices. The entire dosing event may involve several inhalations by the patient with each of the inhalations being provided with drug from the device.

The terms “aerosol,” “particles,” “aerosol particles,” “aerosolized formulation” and the like are used interchangeably herein and shall mean particles of formulation comprised of pharmaceutically active drug and carrier which are formed for aerosol delivery, e.g. upon forcing the formulation through a nozzle which nozzle can be in the form of a flexible porous membrane or generated using a jet or ultrasonic nebulizer. In some embodiments, the particles have a size in the range of 0.5 micron to 12 microns (such as from 1-3.5 microns). In some embodiments, the mean particle size can be within a narrow range such that 80% or more of the particles being delivered to a patient have a particle diameter which is within ±20% of the average particle size, such as ±10% or ±5% of the average particle size.

The terms “particle diameter” and “diameter” are used when referring to the diameter of an aerosol particle and are defined as the “aerodynamic diameter”. The “aerodynamic diameter” is the physical diameter of a sphere of unit density (1 gm/cm³) that has the same terminal sedimentation velocity in air under normal atmospheric conditions as the particle in question. This is pointed out in that it is difficult to accurately measure the physical diameter of small particles using current technology and because the shape may be continually changing. In addition, the deposition of aerosol particles in the bronchial airways of a human subject is described by a Stokes impaction mechanism which is characterized by a particles aerodynamic diameter. Thus, the diameter of one particle of material of a given density will be said to have the same diameter as another particle of the same material if the two particles have the same terminal sedimentation velocity in air under the same conditions.

The term “bulk flow rate” shall mean the average velocity at which air moves through a channel considering that the flow rate is at a maximum in the center of the channel and at a minimum at the inner surface of the channel.

The terms “ambient conditions,” “ambient temperature,” “ambient relative humidity” refer to the conditions of the air surrounding the subject and aerosol production device, prior to this air being entrained into the device and being conditioned by the temperature controller.

The term “temperature sensor” refers to an electrical component that has some measurable, repeatable property that can be used to determine the temperature of the component, and thus the temperature of some other substance which the sensor is in thermal contact with, such as a heating element or the surrounding air. The temperature sensor can be a thermocouple, a diode, or preferably a resistance device such as a thermistor or RTD.

The term “control circuit” refers to a circuit which can perform a number of functions, for example, control of the aerosol production unit, coordination of the sensor with the activation of the aerosol production unit and activation of the heating element, and measuring and controlling the temperature of the heating element. The control sensor can in some embodiments also monitor the temperature and relative humidity of the ambient air, and in some embodiments, can control preheating of the heating element. The control circuit may be an analog circuit, digital circuit, or hybrid analog/digital circuit, and can also include a microprocessor. The control circuit of the invention can be designed to add the desired amount of heat depending on the amount of carrier in the aerosol particles and (1) the density (number of aerosol particles per liter of air) of the generated aerosol (2) the size of the particles initially as well as (3) the size of the particles desired after the carrier has been evaporated away. The control of the aerosol generation device may, for example, share the microprocessor of which the microprocessor may be the type disclosed in U.S. Pat. Nos. 5,404,871, 5,542,410 and 5,655,516.

### Devices

An aerosol dispensing device for use in conjunction with an aerosol production unit for the delivery of drugs via aerosol to the lung is disclosed. The aerosol dispensing device comprises a mechanical burst valve, which is configured to prevent airflow through the device until the subject's inhalation produces a sufficient operational pressure difference across the burst valve. The burst valve can be located in a channel of the aerosol dispensing device which forms an air flow path through the device. The channel can extend from a distal end comprising a first opening into which ambient air can be drawn to a proximal end with a second opening which comprises a mouthpiece. The driving force for the air flow through the channel of the aerosol dispensing device is the subject's inspiratory effort.

The mechanical burst valve is configured such that once a desired operational pressure difference is reached, the burst valve will mechanically open. As such, the burst valve can be configured to determine the initial operational pressure difference and thereby the initial air flow rate of the aerosol dispensing device. In this manner, the drug can be released at a consistent initial air flow rate to ensure repeatability of dosing, and also ensures that a high percentage of the drug released is delivered to a subject.

Other mans may be employed to control the upper limit air flow rate so that a too high air flow rate is not employed after opening of the mechanism burst valve; e.g., a flow restrictor located upstream from the burst-valve. A narrow operational range in air flow rate leads to less inter- and intra-subject variation in the dose that reaches the airways.

In a liquid-based aerosol delivery device where powder clumping is not a problem, the burst valve can be designed to open at a relatively low pressure differential. A low operating pressure differential can make it easier for subjects to inhale through the device. The operational pressure differential can in some embodiments be less than 20 cm H₂O, such as 15 cm H₂O or less, or 10 cm H₂O or less. In some embodiments, the operational pressure difference across the burst valve can be 5 to 15 cm H₂O, or 10 cm H₂O. The operational pressure difference of the burst valve can be set equal to the pressure differential of the entire system, under the desired flow rate. For example, if drawing 60 liters per minute (LPM) of air through the entire system creates a differential pressure (pressure drop) of 10 cm H₂O, and if the burst valve is designed to open at 10 cm H₂O, then by default the initial flow will be 30 LPM. The instantaneous flow rate when the burst valve opens can in some embodiments be in the range of 20-40 liters/min, such as from 25-35 liters/min, or from 30-35 liters/min.

The burst valve can be any suitable mechanical burst valve, such that the burst valve is configured to prevent airflow through the device until the subject's inhalation produces a sufficient operational pressure difference across the burst valve. As such, the burst valve can be a check valve (one-way valve), such as a swing check valve, which can have a flap configuration or disc configuration of the same diameter as the air flow channel, or a lift check valve (which can be a piston or cone shape), a diaphragm check valve, etc. In some embodiments, the moving portion of the valve can be of the same diameter as the air flow channel, and in other embodiments the moving portion of the valve can be smaller than the diameter of the air flow channel, e.g., a portion of the valve may be fixed in position, as with a piston valve. The valve may be fixed to the air flow channel at one point (e.g., as with a hinge) or at more than one point. The valve can have any suitable configuration as long as the valve configuration does not significantly restrict the free flow of air in the direction of inhalation. In some embodiments the burst valve can be configured to be an in-line valve, i.e., in which the inlet (e.g., the distal opening) and outlet (e.g., the mouthpiece at the proximal end) are on the same axis as the valve, or in other embodiments the valve can be configured to be at right angles to the axis of the air flow channel.

The burst valve of the subject invention can have a securing mechanism configured to hold the valve closed until the operational pressure difference is reached. For example, since low pressure differentials can be used, in some embodiments the securing mechanism can be a relatively inexpensive permanent magnet attracting a flap of the burst valve. In some embodiments, the flap of the burst valve can be completely or partially constructed of metal, or the flap can have a metal feature attached to a non-metallic flap. In this embodiment, the burst valve can include a permanent magnet which holds a flap of the burst valve closed until the subject inhales to produce the operational pressure difference sufficient to open the valve. In other embodiments, the securing mechanism can be a lever, or any other suitable securing mechanism. The valve can be designed such that it can return to the original closed position when air flow stops, i.e., by design, by weighting, by a spring device, mechanical device, etc. Consistent positioning of the securing mechanism (e.g., a magnet) is important to keep the operating pressure differential consistent. The securing mechanism can be positioned by any suitable means, (e.g., an adhesive to attach the magnet) such that the securing mechanism consistently contacts the valve (e.g., a metal flap) when it is in the closed position.

The mechanical burst valve can further include one or more sensors, which can be configured to sense the position of the burst valve. For example, a sensor can be positioned so that the burst valve can be detected in the open position. In some embodiments, a sensor can detect the burst valve in the closed position. The sensor can be any suitable sensor, such as a mechanical switch, Hall effect sensor, optical switch, etc. In one embodiment, the sensor can be a simple mechanical leaf spring. The sensor (e.g., a switch) can be configured for automatically releasing or triggering the aerosol production unit (e.g., activating a mechanical means, or activating a control circuit) after the inspiratory flow reaches a threshold level (e.g., the burst valve is in the open position). The sensor can therefore be configured such that it can trigger the aerosol production unit to produce an aerosol from a liquid containing a pharmaceutically active drug once the burst valve is in the open position. Activating the aerosol production unit and thereby generating the aerosol only after the burst valve has been opened maximizes the efficiency of aerosol delivery, with minimum wasting of the dose (e.g., by aerosol droplets deposited on the sides of a chamber). The sensor can also be configured such that it can activate a heating element once the burst valve is in the open position (e.g., activating a mechanical means, or activating a control circuit)

The burst valve of the subject invention can determine the initial pressure drop and therefore the flow rate across the valve. However, maintenance of the flow rate depends on the subject's sustained inspiratory effort. Therefore, the aerosol dispensing device can be designed such that the device can vibrate or whistle if the flow rate or pressure is outside of a desired range over the dosage period, i.e., the period during which the drug being inhaled. Other forms of feedback that can be included in some embodiments of the device are other auditory or visual indicators (e.g., audible signal such as an alarm, or an indicator light, etc.). In this manner, the device can assist a subject in maintaining a proper flow rate which is important in the effective delivery of the entire inhaled dose of medication.

The aerosol production unit of the aerosol dispensing device is configured to produce an aerosol from a liquid, the liquid containing a pharmaceutically active drug. The aerosol producing unit can form the aerosol in a chamber of the air flow channel through an opening in the aerosol dispensing device which communicates with the aerosol producing unit. The aerosol can thus be entrained in the airflow and provided to the subject. As discussed above, the aerosol production unit can also be configured such that it is activated to produce an aerosol from a liquid containing a pharmaceutically active drug by the opening of the burst valve as sensed by a sensor, for example, by using a control circuit.

The aerosol production unit can be any suitable system that generates aerosols from liquid formulations which contain a pharmaceutically active drug. The drug can be present in a dosage form, which can be any type of container that holds the liquid to be aerosolized. In some embodiments, the dosage form containing the pharmaceutically active drug can be present as a blister in a blister pack. The dosage form can include nozzles through which the liquid passes under pressure to form liquid jets, which break up into aerosol particles. In this embodiment, mechanical pressure on the blister can cause the liquid to be forced through the nozzle region. The nozzles can be sized to form relatively small aerosol particles, e.g. one micron in diameter at the exit. One embodiment of a dosage form has an array of nozzles. In some embodiments, a peel channel can be used to pass the liquid to the nozzle region under pressure.

The aerosol production unit can include an aerosol creating mechanism to create the aerosol from the liquid, e.g., a plunger or piston. In one embodiment, the aerosol creating mechanism can comprise a plunger that strikes into a container of the liquid when the burst valve opens to create the aerosol. In some embodiments, the aerosol creating mechanism can be configured such that it is activated to produce an aerosol from a liquid containing a pharmaceutically active drug by the opening of the burst valve as sensed by a sensor, for example, by using a control circuit.

The aerosol generation device of the present invention can be loaded with a disposable dosage form of the type disclosed within U.S. Pat. Nos. 5,497,763, 5,544,646, 5,660,166 and 5,718,222, all of which are incorporated herein by reference to disclose an aerosol generation device and a disposable container for containing a drug for aerosolized delivery. The dosage form can be part of a strip of dosage forms that can be automatically or manually advanced to replace a used dosage form in position at the nozzle slot.

The pharmaceutically active drug which is administered to the subject via inhalation may be in a variety of different compositions. For example, the drug can be an aqueous solution of drug suspended in or dissolved in a carrier. Carriers can include water, ethanol and mixtures thereof. The liquid formulation of the pharmaceutically active drug can be formed into small particles by the aerosol generation unit which is delivered to the subject. In some embodiments, the drug may be in a solution wherein a low-boiling point propellant is used as a solvent.

The amount of drug or dosage delivered to the subject can vary greatly depending on the particular drug being delivered. A wide range of drugs can be delivered, including, for example, hormone drugs. For example, drugs delivered could be drugs which have a systemic effect e.g., leuprolide, insulin and analogs thereof including monomeric insulin, or morphine; or a local effect in the lungs e.g., Activase t-PA, albuterol, or sodium cromoglycate.

The aerosol dispensing device can in some embodiments include a heating element, which is configured to evaporate water from aerosol droplets created by the aerosol production unit. The heating element can be configured such that it is activated by the opening of the burst valve as sensed by the sensor. For example, the heating unit can turn on once the burst valve is in the open position. The heating element can be used to ensure a more uniform particle size and thus a more consistent level of drug delivery. The aerosol dispensing device can in some embodiments also include a temperature sensor. Correct operation of the heating element depends on the flow rate, because if the flow rate is too slow, this can result in too much energy provided to the aerosol droplets. As discussed above, the burst valve can ensure that air flows that are too slow do not occur. A heating element of one embodiment is described in the U.S. Pat. 6,845,216, hereby incorporated by reference.

The aerosol dispensing device can also have a portable power unit such as a battery. Any suitable type of battery can be used, including standard sized cells, such as AA size batteries. The batteries can be disposable or rechargeable.

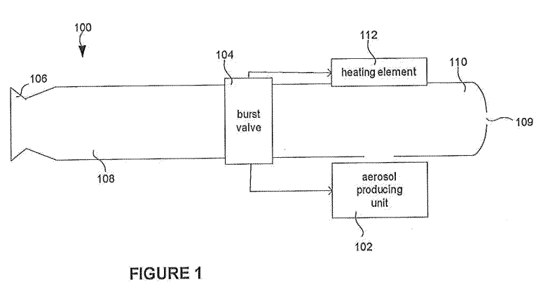

FIG. 1 shows an example of an aerosol delivery device 100 of the subject invention. The aerosol dispensing device has a channel which forms an air flow path through the device. The channel extends from the distal end with a first opening 109 into which ambient air can be drawn to a proximal end with a second opening which comprises a mouthpiece 106. The aerosol delivery device 100 can include an aerosol producing unit 102 which can produce an aerosol from a liquid. The liquid can contain a pharmaceutically active drug for dispensing to a subject. The burst valve 104 can be adapted to mechanically open in response to a subject's inhalation. The burst valve 104 located in air flow path can prevent airflow through the device 100 until the subject's inhalation produces an operational pressure difference across the burst valve 104.

In using the device, the subject can inhale at a mouthpiece 106. The inhaled air can enter through opening 109, and flow through chamber 110, the opened burst valve 104, and air chamber 108 before passing through the mouthpiece 106 to the subject. The aerosol producing unit 102 can form the aerosol in chamber 110. From chamber 110, the aerosol can be entrained in the airflow and thus provided to the subject.

As discussed above, the operational pressure differential across the burst valve can be set relatively low, for example, in one example, the operational pressure difference can be set at 10 cm H₂O. An operational pressure difference of 10 cm H₂O might correspond to an air flow rate or bulk flow rate for example of 30 LPM, which will ensure that the initial flow through the device is 30 LPM. Providing a sufficiently high flow rate through the device during operation is important, as for example, a flow rate that is too slow can lead to the aerosol being deposited on the sides of the chamber 108 and 110, and thus reducing the amount of aerosol delivered to the patient as aerosol. Designing the system to engage at a flow rate that is too high may result in greater orpharyngeal deposition due to aerosol particles being imparted with greater momentum, and thus a reduction in delivery to the airways and/or the peripheral lung.

In one embodiment, the burst valve 104 is designed such that the instantaneous flow rate when the burst valve opens is in the range of 20-40 liters/min, however any desired flow rates can also be used.

The invention makes it possible to obtain a higher degree of repeatability in dosing with an aerosol device. By using the burst valve on the aerosolized drug delivery device it is possible to set the valve at a given level for release of drug and thereafter always have the drug aerosolized and released to the patient at the same patient inhalation rate. If timing is also taken into consideration then the drug can be released at the same inspiratory flow rate and inspiratory volume. When the drug is administered the next time the patient then again releases the aerosolized drug at the same inspiratory rate and inspiratory volume as was performed during the first drug delivery event. This sequence is repeated time and again over a period of days, weeks, months or years or even more. This makes it possible to increase the repeatability of dosing delivered to the patient.

A heating element 112 is also provided, which can be used to ensure a more uniform particle size and thus a more consistent level of drug delivery. The heating element can be connected to a self-contained power source (e.g., a battery, not shown). In some embodiments, the power source may be a container of a liquid substance such as butane or propane. The aerosol production unit 102 and heating element 112 can be triggered by the opening of the burst valve 104.

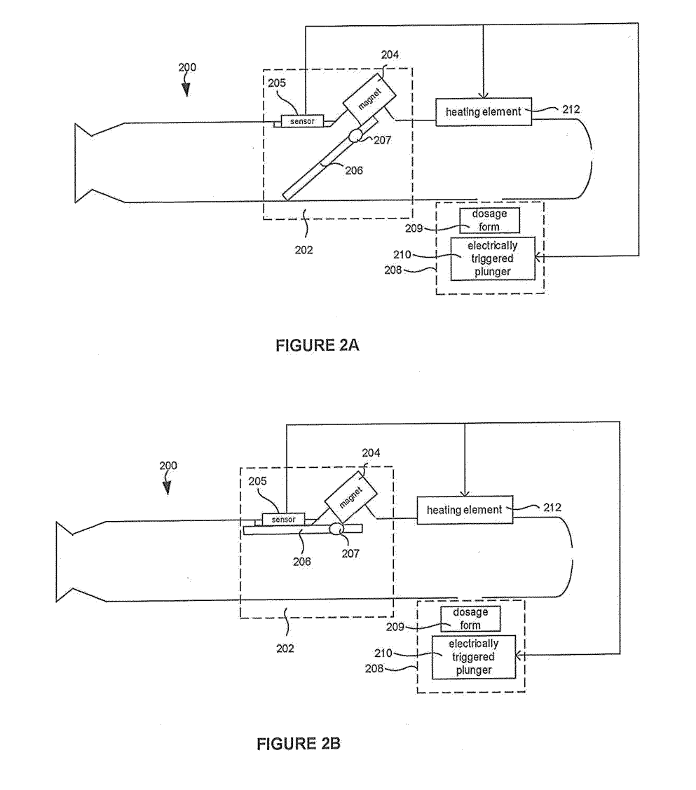

FIGS. 2A and 2B illustrate the operation of one embodiment of an aerosol dispensing device 200. FIG. 2A shows the closed state of the burst valve 202; FIG. 2B shows the open state of the burst valve 202. In this embodiment, the burst valve 202 includes a permanent magnet 204 which holds a flap 206 of the burst valve closed until the subject inhales to produce the operational pressure difference of the burst valve. In one embodiment, the flap 206 can be wholly or partially made of metal. In this illustration, the flap 206 is shown as rotating about hinge 207.

The sensor 205 can be configured such that it can trigger the aerosol producing unit 208 to produce an aerosol from a liquid containing a pharmaceutically active drug once the burst valve is in the open position. The aerosol producing unit 208 can include a plunger 210 that strikes into a container of the liquid to create the aerosol when the burst valve 202 opens. A dosage form 209, such as a blister pack, can contain the liquid to form the aerosol. The plunger 210 can strike the liquid in dosage form 209 when the burst valve opens to create the aerosol.

The sensor 205 can also be configured such that it can activate heating element 212 once the burst valve is in the open position. The heating element 212 can turn on when the burst valve opens to evaporate liquid from the aerosol droplets.

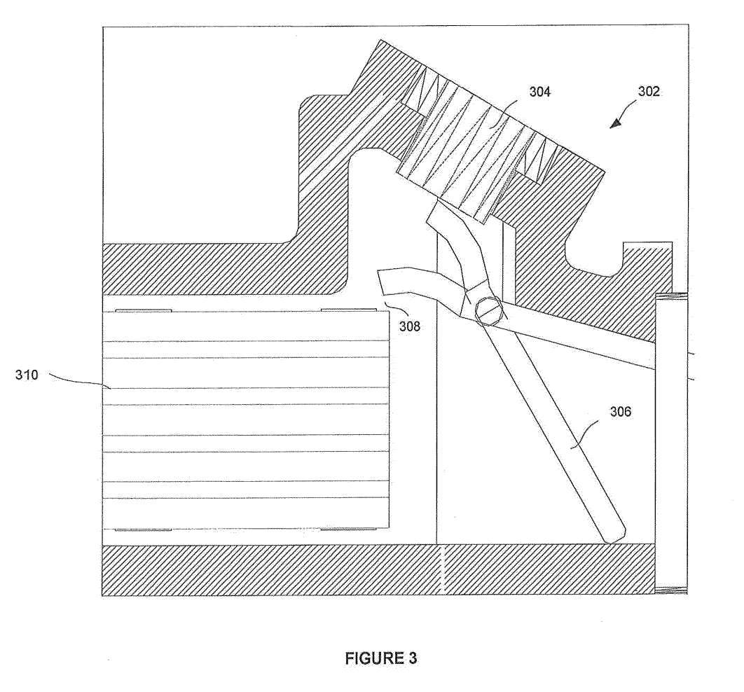

FIG. 3 shows a detail of a burst valve 302 with permanent magnet 304 and metal flap 306. Other mechanical burst valve designs can also be used.

As shown in FIGS. 2A and 2B, a sensor (not shown) can be positioned in region 308 so that the open position of the burst valve can be detected. FIG. 3 also shows a heating element 310.

### Methods

Aspects of the invention include methods of treating a subject by administering an aerosolized liquid formulation comprising a pharmaceutically active drug. The methods can include administering any suitable pharmaceutically active drug using the aerosol delivery devices of the subject invention, as described above. In the subject methods, an aerosol delivery device can be used which comprises a burst valve that is configured to prevent airflow through the device until the subject's inhalation produces an operational pressure difference across the burst valve. Therefore, the methods can include administering the drug by inhalation when a burst valve is mechanically opened, wherein the burst valve is configured to prevent airflow through the device until the subject's inhalation produces an operational pressure difference across the burst valve. In some embodiments the operational pressure difference is 15 cm H₂O or less, including between 5 and 15 cm H₂O, or 10 cm H₂O.

The term “treatment” or “treating” is used here to cover any treatment of any disease or condition in a mammal, particularly a human, and includes: (a) preventing the disease or condition from occurring in a subject which may be predisposed to the disease but has not yet been diagnosed as having it; (b) inhibiting the disease or condition, i.e. arresting its development; and/or (c) relieving the disease or condition, i.e. causing regression of the disease and/or its symptoms.

Administering of an aerosolized liquid formulation comprising a pharmaceutically active drug can include the administration of one or more doses of one or more pharmaceutically active drugs, which can be administered to treat the lung locally, or can be administered as a systemic drug.

### Examples

The following examples are put forth so as to provide those of ordinary skill in the art with a complete disclosure and description of how to make and use various constructs and perform the various methods of the present invention and are not intended to limit the scope of what the inventors regard as their invention nor are they intended to represent or imply that the embodiments described below are all on the only embodiments constructed or tested. Efforts have been made to ensure accuracy with respect to numbers used (e.g., amounts, concentrations, particular components, etc.) But some deviations should be accounted for.

### Example 1

A subject in need of treatment is provided. An aerosol dispensing device comprising a mechanical burst valve which is set to open at an instantaneous flow rate when the burst valve opens of approximately 30 liters per minute (LPM) is used by the subject. Upon inhaling with sufficient force, the mechanical burst valve opens. A sensor comprising a permanent magnet senses the open position of the burst valve, and activates the aerosol producing unit to produce the aerosol, by activating a plunger which strikes the liquid in a blister container. The sensor also activates a heating element. The subject maintains an air flow sufficient for the intrapulmonary delivery of the aerosol by viewing an indicator light, which remains lit while the burst valve is open; e.g. when the flow rate is at least 30 LPM. The subject maintains sufficient force for approximately 5 seconds at which point the dose of aerosolized drug has been administered.

This drug delivery method can be repeated a number of times in order to obtain repeatable dosing. Those skilled in the art will understand that drug delivery by inhalation has a number of desirable characteristics including the ability to obtain almost immediate blood levels of the drug which is a substantial improvement over oral delivery. However, even though the speed of delivery of the drug to the blood stream is substantially the same as that with injection the repeatability of the dosing is often less precise as compared with injection. The present invention improves that repeatability by making it possible to repeatedly deliver aerosolized medication to a patient at substantially the same inspiratory flow rate and inspiratory volume time and again.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalents.

What is claimed is:

1. An aerosol dispensing device comprising:
a burst valve configured to mechanically open in response to a subject's inhalation, wherein the burst valve is configured to prevent airflow through the device until the subject's inhalation produces an operational pressure difference across the burst valve;
an aerosol production unit configured to produce an aerosol from a flowable formulation of a pharmaceutically active drug.

2. The aerosol dispensing device according to claim 1, wherein the operational pressure difference is 15 cm H₂O or less,
wherein the burst valve comprises a mechanism configured to hold the valve closed until the operational pressure difference is reached.

3. The aerosol dispensing device according to claim 2, wherein the operational pressure difference is between 5 and 15 cm H₂O,
wherein the mechanism configured to hold the valve closed comprises a magnet.

4. The aerosol dispensing device according to claim 3, wherein the operational pressure difference is 10 cm H₂O,
wherein the burst valve comprises a flap configured to be held closed by the magnet.

5. The aerosol dispensing device according to claim 1, wherein the burst valve further comprises a sensor and the formulation is a dry powder.

6. The aerosol dispensing device according to claim 5, wherein the aerosol production unit is activated by the opening according to the burst valve as sensed by the sensor and the formulation is a liquid present in a container.

7. The aerosol dispensing device according to claim 6, wherein the aerosol production unit includes a plunger that strikes into the container according to the liquid to create the aerosol when the burst valve opens.

8. The aerosol dispensing device according to claim 6, wherein the container is comprised of a material which is collapsible upon the application of force and further comprised of a porous membrane, the porous membrane having a plurality of pores therein, wherein the pores have a size and configuration which allows for the formation of aerosolized particles having an aerodynamic particle size in a range of from about 0.5 micron to 12 microns.

9. The aerosol dispensing device according to claim 1, further comprising a heating element configured to evaporate water from aerosol droplets and wherein the burst valve further comprises a sensor and the heating element is activated by the opening according to the burst valve as sensed by the sensor.

10. A method of treating a subject comprising:
(a) aerosolizing a formulation of flowable material comprising a pharmaceutically active drug;
(b) administering the drug by inhalation when a burst valve is mechanically opened, wherein the burst valve is configured to prevent airflow through the device until the subject's inhalation produces an operational pressure difference across the burst valve; and
(c) repeating the aerosolizing (a) and (b) administering a plurality of times.

11. The method according to claim 10, wherein the operational pressure difference is 15 cm H₂O or less wherein the burst valve comprises a securing mechanism configured to hold the valve closed until the operational pressure difference is reached wherein the burst valve comprises a flap configured to be held closed by the magnet.

12. The method according to claim 11, wherein the operational pressure difference is between 5 and 15 cm H₂O wherein the mechanism configured to hold the valve closed comprises a magnet.

13. The method according to claim 12, wherein the operational pressure difference is 10 cm H₂O.

14. The method according to claim 10, further comprising:
sensing the position of the burst valve with a sensor wherein the aerosolization of the liquid is activated by the opening of the burst valve as sensed by the sensor.

15. The method according to claim 10, wherein the flowable material is present in a container and the material is chosen from a flowable powder and a liquid.

16. The method as claimed in claim 15, wherein the flowable material is a liquid and the liquid is present in a container, the method further comprising:
applying pressure to the container in order to force the liquid through a porous membrane of the container and form aerosolized particles having a particle diameter in a range of 0.5 micron to 12 microns.

17. The method as claimed in claim 10, further comprising:
heating the aerosolized formulation in a manner so as to evaporate away excipient material in the formation.
